(19) United States
(12) Patent Application Publication
(10) Pub. No.: US 2026/0345893 A1
(43) Pub. Date: Nov. 5, 2026

(54) INTRAVENOUS LINE FLUSHING DEVICE

(71) Applicant: CareFusion 303, Inc., San Diego, CA (US)

(72) Inventors: Praveen NALAWADE, Bengaluru (IN); Shishir PRASAD, Ramsey, NJ (US); Rahul Rajan PUTHUKKAD, Bengaluru (IN); Shashwat JAIN, Indore (IN)

(21) Appl. No.: 19/197,788

(22) Filed: May 2, 2025

(51) Int. Cl. A61M 5/14; A61M 39/00; A61M 39/24

(52) U.S. Cl. CPC A61M 5/1413; A61M 39/24; A61M 2039/0018; A61M 2039/2493; A61M 2202/04; A61M 2205/0216; A61M 2205/3331

(57) ABSTRACT

Flushing devices can be desirable to efficiently flush the IV line and can be coupled to an IV set between a fluid source and a drip chamber or IV tubing. Medication flows from the fluid source, through a flow chamber of the flushing device, and into the rest of the IV line. The flushing device can also include or be coupled to a saline source, which fills a saline chamber of the flushing device with saline. The saline chamber is connected to the flow chamber such that the saline only enters the flow chamber and, thus, the IV line, after the medication enters the IV line.

### TECHNICAL FIELD

The present invention relates generally to devices and methods for flushing an intravenous (IV) line and, in particular, to flushing devices that can be coupled to an IV set to selectively direct one or more fluid therethrough.

### BACKGROUND

Medical treatments often include the infusion of a medical fluid (e.g., a saline solution or a liquid medication) to patients using an IV catheter that is connected though an arrangement of flexible tubing and fittings, commonly referred to as an “IV set,” to a source of fluid, for example, an IV bag.

During IV fluid delivery, between about 10% and 32% of the medical fluid can become trapped with the IV set due to dead space within the IV set. Consequently, the medical fluid does not reach the patients, which increases the risk of treatment failure due to underdosing, increases the average length of hospital stays (which poses a burden to caregivers), accelerates antimicrobial resistance, and wastes medication. In some instances, for example, at least 20% of medical fluid can become trapped in an IV set and discarded as clinical waste.

### SUMMARY

With conventional IV fluid delivery, caregivers access medical fluid that is trapped in an IV set by manually flushing the IV line. However, this is very time consuming for caregivers, who are often overburdened. Therefore, an IV line flushing device that limits the amount of medical fluid trapped inside an IV set can be desirable to mitigate health risks to patients receiving infusion treatment, reduce the workload of caregivers who are manage the patients'infusion treatment, and limit the amount of medical fluid that is wasted. In accordance with this realization, some of the embodiments in the present disclosure are directed to a flushing device that automatically flushes the IV line after a full dose of a medical fluid enters the IV line for delivery to a patient. Other embodiments in the present disclosure are directed to a flushing device that can be used to prime the IV line before fluid delivery, in addition to flushing the IV line after fluid delivery. Moreover, the present disclosure is also directed to a flushing device that limits the mixture of the medical fluid (i.e., the drug) with the flushing fluid (i.e., saline), which reduces the occurrence of dilution and facilitates efficient IV fluid delivery.

Accordingly, some embodiments of the present disclosure are directed to a flushing device for an intravenous (IV) line, the flushing device comprising an inlet port configured to fluidly connect the flushing device to a fluid source, an outlet port configured to fluidly connect the flushing device to the IV line, a flow chamber fluidly connected to the inlet port and the outlet port, a saline chamber separated from the flow chamber by a wall and configured to be filled with saline, one or more openings in the wall, and one or more flaps overlapping each of the one or more openings from inside the flow chamber and movably coupled to the wall, wherein the one or more flaps close each of the one or more openings when a flow chamber pressure is greater than a saline chamber pressure, wherein the one or more flaps expose each of the one or more openings when the flow chamber pressure is less than the saline chamber pressure, such that each of the one or more openings fluidly connects the flow chamber and the saline chamber.

In some embodiments, the outlet port is configured to be coupled to a drip chamber of the IV line.

In some embodiments, each of the one or more openings is configured to facilitate a flow of the saline from the saline chamber to the flow chamber when the flow chamber pressure is less than the saline chamber pressure.

In some embodiments, the flow chamber pressure is less than the saline chamber pressure when the flow chamber is empty and the saline chamber contains at least some of the saline.

In some embodiments, the saline chamber is formed between the wall and an outer wall of the flushing device. Optionally, the flushing device further comprises a stopper that presses the one or more flaps against the wall inside the flow chamber, wherein the stopper is slidably coupled to the wall such that the stopper is configured to slide towards the inlet port and away from the one or more flaps when the IV line is coupled to outlet port. Optionally, the outer wall comprises an elastic material such that the outer wall is configured to bow inwards towards the flow chamber when the flow chamber pressure is less than the saline chamber pressure.

In some embodiments, the flow chamber is adjacent to the saline chamber. Optionally, the flushing device further comprises a saline inlet in the saline chamber and a saline source filled with saline, wherein the saline inlet is configured to fluidly connect the saline source to the saline chamber. Optionally, the saline source comprises a collapsible bag that is configured to collapse as the saline flows from the saline source to the saline chamber when the flow chamber pressure is less than the saline chamber pressure. Optionally, the flushing device further comprises a clamp coupled to the saline inlet, wherein the clamp is configured to fluidly disconnect the saline source and the saline chamber when the IV line is being primed.

Some embodiments of the present disclosure are directed to a flushing device for an intravenous (IV) line, the flushing device comprising: a Y-site connector comprising a first inlet that leads to a flow lumen and is configured to couple with a fluid source and a second inlet that leads to a saline lumen that intersects the flow lumen; and a Tesla valve comprising a downstream end coupled to the second inlet and an upstream end configured to couple with a saline source, wherein the flow lumen is configured to transport fluid from the fluid source to the IV line, wherein the Tesla valve is configured to obstruct a flow of the fluid towards the saline source and limit mixing of the fluid and the saline.

In some embodiments, the flushing device further comprises the saline source, wherein the saline source is configured to be positioned at a first height, the fluid source is configured to be positioned at a second height, and the first height is at or below the second height, such that the saline flows through the Tesla valve when a level of the fluid is at or below the first height.

In some embodiments, the saline lumen is configured to transport saline from the saline source to the IV line after the fluid source is empty.

In some embodiments, the flow lumen has a first diameter, a flow path in the Tesla valve has a second diameter, and the first diameter is larger than the second diameter, such that the Tesla valve is configured to slow the flow of the saline towards the IV line.

Other embodiments of the present disclosure are directed to a flushing device for an IV line, the flushing device comprising an inlet port configured to fluidly connect the flushing device to a fluid source, an outlet port configured to fluidly connect the flushing device to the IV line, a flow channel fluidly connected to the inlet port and the outlet port, a saline channel fluidly connected to the flow channel at an intersection, a saline chamber fluidly connected to the saline channel, and a dial coupled to the flow channel and the saline channel at the intersection and comprising a no flow setting, a flow setting, and a saline setting, wherein when the dial is turned to the no flow setting, the dial obstructs the flow channel to resist fluid from the fluid source from flowing through the flow channel and obstructs the saline channel to resist saline from the saline chamber from flowing into the flow channel, wherein when the dial is turned to the flow setting, the dial permits the fluid from the fluid source to flow through the flow channel towards the outlet port and obstructs the saline channel to resist the saline from flowing into the flow channel, wherein when the dial is turned to the saline setting, the dial obstructs the flow channel to resist the fluid from the fluid source from flowing through the flow channel towards the outlet port and permits the saline to flow from the saline channel into the flow channel towards the outlet port.

In some embodiments, the dial comprises a connecting channel. Optionally, when the dial is turned to the no flow setting, the connecting channel is misaligned with the flow channel and the saline channel. Optionally, when the dial is turned to the flow setting, the connecting channel fluidly connects the flow channel and the outlet port. Optionally, when the dial is turned to the saline setting, the connecting channel fluidly connects the saline channel and the outlet port.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an IV fluid delivery set coupled to a patient, in accordance with aspects of the present disclosure.

FIGS. 2A-2H illustrate an embodiment of the flushing device, in accordance with aspects of the present disclosure.

FIGS. 3A-3E illustrate another embodiment of the flushing device, in accordance with aspects of the present disclosure.

FIGS. 4A-4E illustrate another embodiment of the flushing device, in accordance with aspects of the present disclosure.

FIGS. 5A-5C illustrate another embodiment of the flushing device, in accordance with aspects of the present disclosure.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an intravenous (IV) fluid delivery set, such embodiments can be used in other fluid conveyance systems. Furthermore, various applications of such embodiments and modifications thereto, which may occur to those who are skilled in the art, are also encompassed by the general concepts described herein.

Flushing an intravenous set is an essential step that follows delivering medication to a patient intravenously. For example, after the medication (hereinafter referred to as the medical fluid or the fluid) is in the IV line, a caregiver often flushes the IV line with saline. However, manual flushing is time consuming and can lead to dilution of the medical fluid with saline. Therefore, a device that maintains the separation of the medical fluid and the saline can be valuable. This can be accomplished with a device with separate chambers, channels, lumens, etc. for the medical fluid and the saline, where flow of the saline into the IV line is contingent on all of the medical fluid being transported into the IV line.

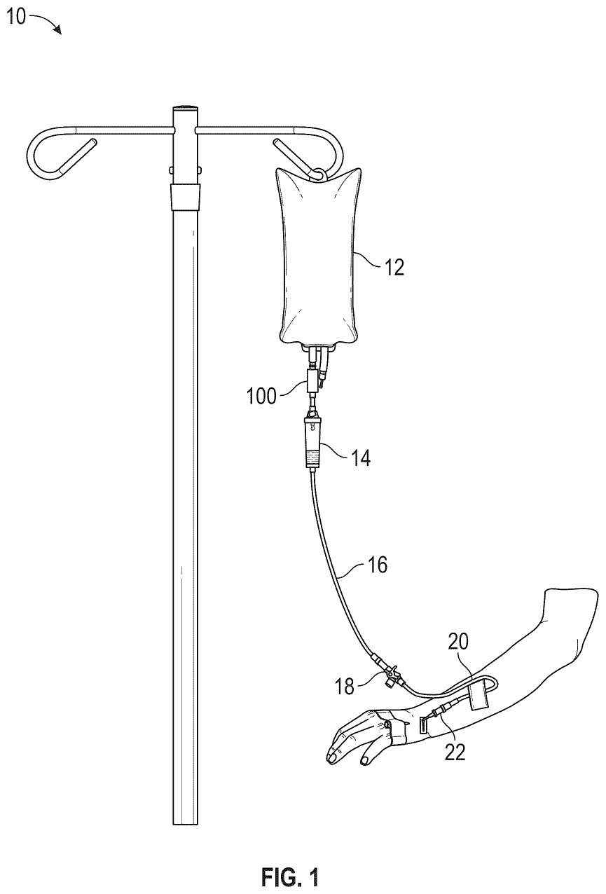

Referring now to the figures, FIG. 1 illustrates an IV fluid delivery set coupled to a patient, in accordance with aspects of the present disclosure. The IV set 10 includes a medical fluid bag 12, an IV line flushing device 100, a drip chamber 14, a first tube 16, a fluid connector assembly 18, a second tube 20, and a catheter 22. The tubing 16 extends between the drip chamber 14 and the fluid connector assembly 18. The tubing 20 extends between the fluid connector assembly 18 and the catheter 22, which is inserted in the arm 50 of the patient.

The flushing device 100 is designed for use in medical applications, such as the IV set 10 as well as other IV medical fluid delivery applications using infusion pumps, as non-limiting examples. The flushing device 100 automatically flushes the IV line (which includes the tubing 16, the connector 18, the tubing 20, and the catheter 22) with saline after the medical fluid bag 12 is empty (i.e., after the patient has received the full dose of medication from the medical fluid bag 12).

The present disclosure illustrates and describes various embodiments of the flushing device. For example, the flushing device can include a flow chamber. The flow chamber has an inlet port for connecting the flushing device to a fluid source (such as an infusion pump, an IV bag, or the medical fluid bag 12 in FIG. 1). The flow chamber also has an outlet port for connecting the flushing device to a drip chamber or an IV line (such as the drip chamber 14 or the tubing 16 in FIG. 1). The flow chamber of the flushing device receives a medical fluid from the fluid source via the inlet port, and the medical fluid flows through the flow chamber and out the outlet towards the patient.

The flushing device can also include a saline chamber, which is filled with saline. The saline chamber is separated from the flow chamber by a wall. There is at least one opening in the wall, and each opening in the wall is covered by a flap. Each flap is movably coupled to the wall from inside of the flow chamber such that each flap covers its corresponding opening to close the opening and can move away from its corresponding opening to expose the opening, at which point saline can flow from the saline chamber and into the flow chamber via the opening(s). Whether the flap covers or moves away from its corresponding opening depends on the relative pressures in the flow chamber and the saline chamber. When the pressure in the flow chamber is greater than the pressure in the saline chamber, each flap is pressed against the wall to cover and close the opening. On the other hand, when the pressure in the flow chamber is less than the pressure in the saline chamber, the movement of each flap is unrestricted. Additionally, because the pressure in the flow chamber is less than the pressure in the saline chamber, saline will flow towards the flow chamber. As a result, the saline will push each flap away from its corresponding opening and enter the flow chamber.

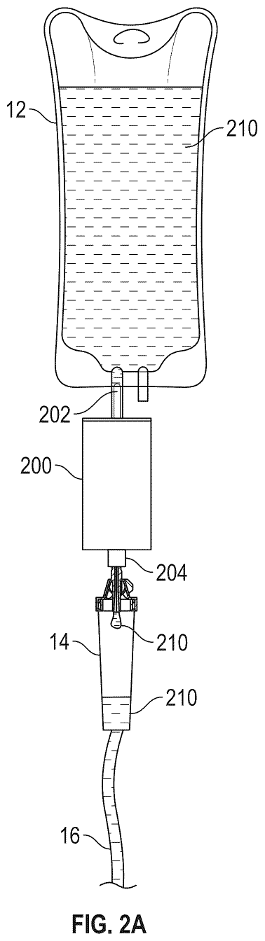

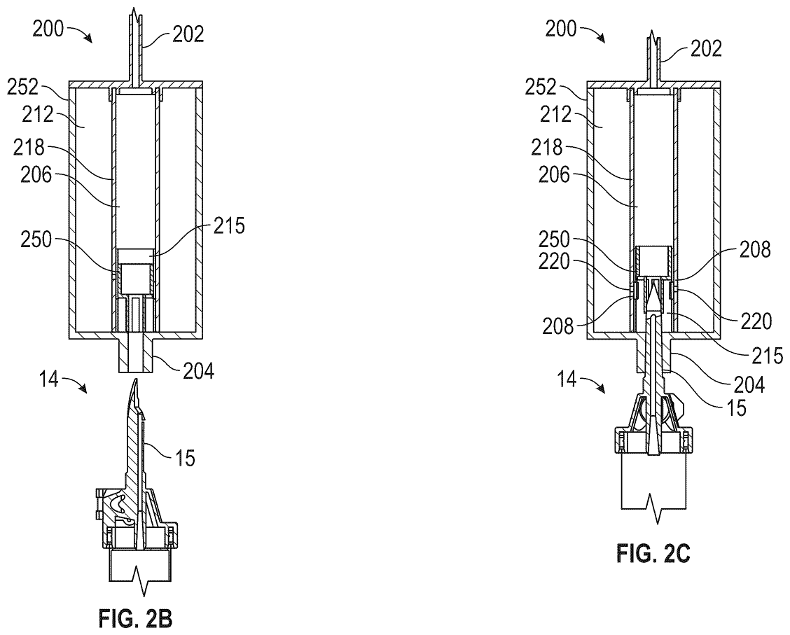

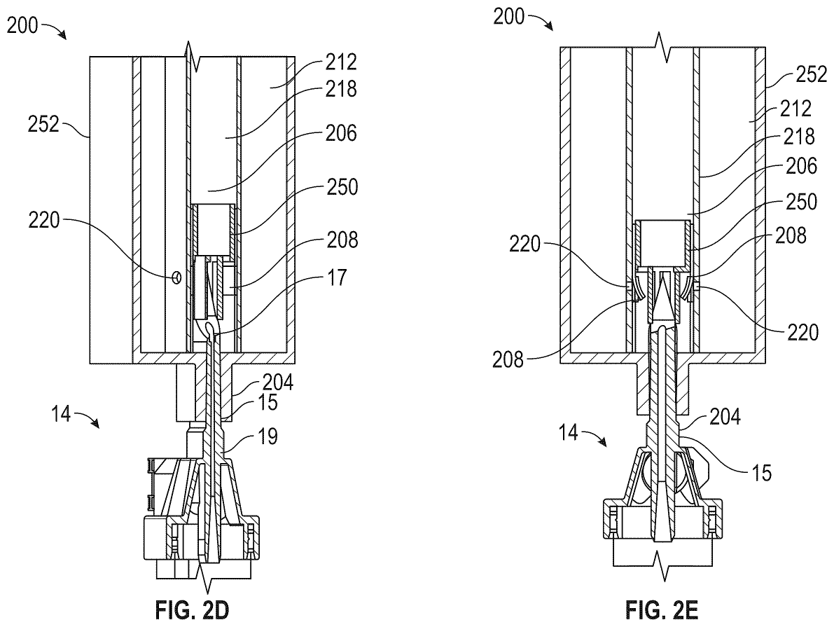

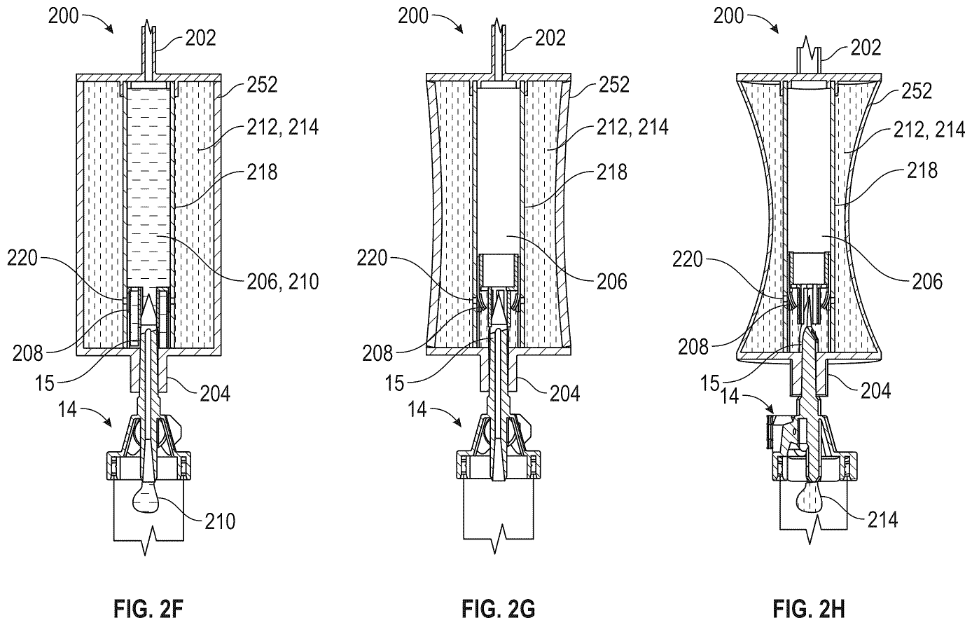

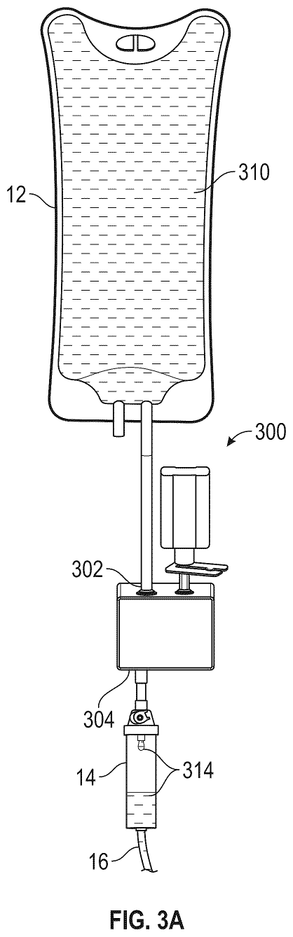

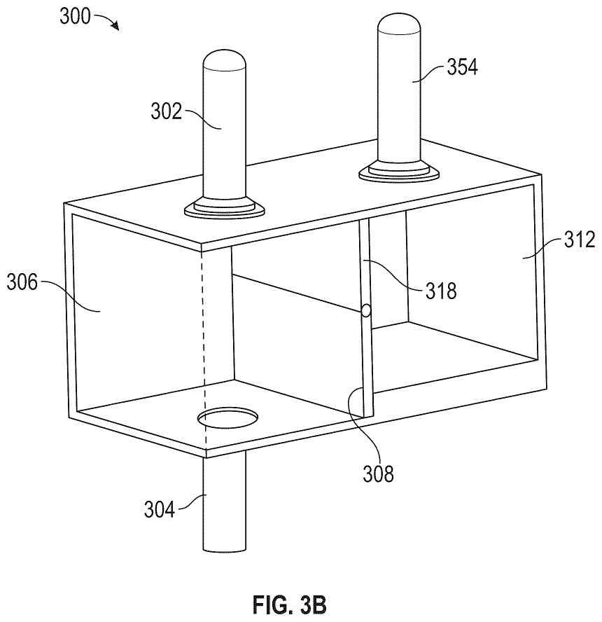

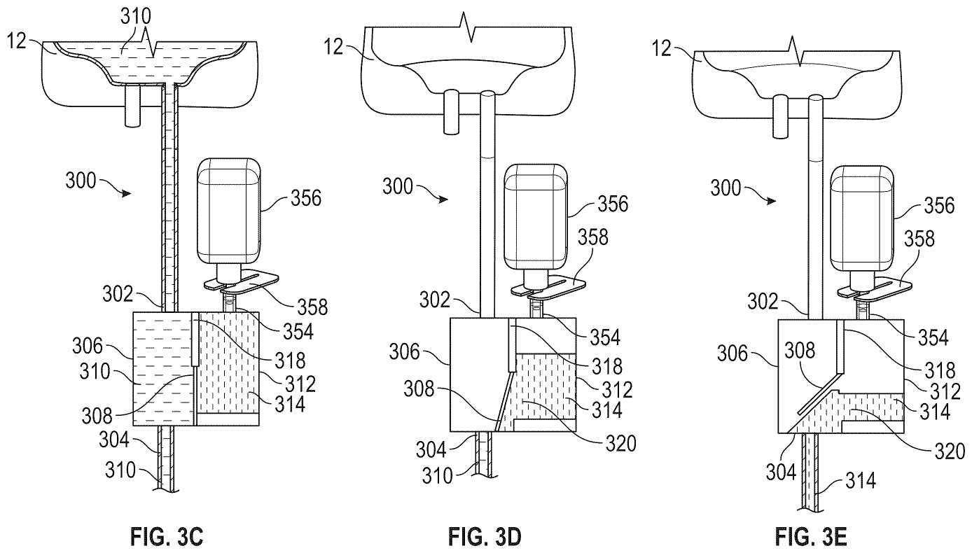

The pressure in the flow chamber is less than the pressure in the saline chamber when there is less medical fluid in the flow chamber than saline in the saline chamber, which would generally occur after the fluid source is empty. Accordingly, the flushing device automatically flushes the IV line, because when the fluid source is empty (i.e., after IV delivery of the medical fluid is complete), the pressure in the flow chamber is less than the pressure in the saline chamber, so the saline flows towards the flow chamber. After the saline enters the flow chamber, the saline flows out the outlet port and towards the remainder of the IV line to flush the IV line. FIGS. 2A-3E depict two examples of this flushing device embodiment.

FIGS. 2A-2H illustrates a first example of the flushing device, in accordance with aspects of the present disclosure. As shown in FIG. 2A, the flushing device 200 can be coupled to an IV line between a medical fluid bag 12 and a drip chamber 14. The flushing device 200 has an inlet port 202 that fluidly connects the flushing device 200 to the medical fluid bag 12. In some embodiments, the inlet port 202 is a spike configured to spike the medical fluid bag 12. The fluid 210 from the medical fluid bag 12 can flow through the inlet port 202, into the flushing device 200, and out the outlet 204 on the other side of the flushing device 200. In the embodiment shown, the outlet 204 is configured to couple the flushing device 200 to the drip chamber 14. In some embodiments, the outlet 204 can be coupled directly to an IV tube 16.

FIG. 2B depicts the flushing device 200 before it is coupled to the drip chamber 14. The flushing device 200 has a flow chamber 206 and a saline chamber 212. As shown, the flow chamber 206 is positioned within the saline chamber 212. In some embodiments, the flow chamber 206 and the saline chamber 212 are concentric cylinders. In other embodiments, the flow chamber 206 and the saline chamber 212 are nested rectangular vessels. Optionally, the flow chamber 206 and the saline chamber 212 have different shapes (e.g., the flow chamber 206 is cylindrical and the saline chamber 212 is rectangular).

The saline chamber 212 is fluidically separated from the flow chamber 206 by a wall 218. In accordance with the description of the wall, the opening(s), and the flap(s) described above, the wall 218 includes openings 220 that are covered by flaps 208, which are movably engaged against the wall 218 to permit or resist movement of a fluid between the saline chamber 212 and the flow chamber 206. In some embodiments of the present disclosure, the flaps 208 can be coupled to the wall 218 and can move toward or away from an inside surface of the flow chamber 206 to permit or resist movement of a fluid between the saline chamber 212 and the flow chamber 206. In some embodiments, the flaps 208 engage against the wall 218 to obstruct the openings 220 to resist movement of a fluid between the saline chamber 212 and the flow chamber 206.

The flushing device 200 illustrated in FIGS. 2A-2H also has a stopper 250 positioned inside the flow chamber 206 and configured to slide within the flow chamber 206 relative to the flaps 208. Before the flushing device 200 is coupled to the drip chamber 14, the stopper 250 is in a first position, shown in FIG. 2B, in which the stopper 250 is axially aligned with the flaps 208. In the first position, the stopper 250 is presses the flaps 208 against the wall 218, thereby resisting any movement of the flaps 208 away from the openings 220 and keeping the openings 220 closed. However, when the flushing device 200 is coupled to the drip chamber 14, the spike 15 of the drip chamber 14 slides the stopper 250 into a second position, shown in FIG. 2C. In the second position, the stopper 250 is axially offset from the flaps 208, such that the flaps 208 can move away from the openings 220, thereby fluidly connecting the saline chamber 212 and the flow chamber 206.

In some embodiments, the flushing device 200 also includes a sleeve 215 inside the flow chamber 206, and the stopper 250 is slidably coupled to the sleeve 215 instead of the wall 218. In those embodiments, the flaps 208 can be movably coupled to the sleeve 215, and the openings 220 extend through both the wall 218 and the sleeve 215.

FIG. 2C illustrates the flaps 208 that cover the openings 220, and FIG. 2D illustrates a perspective view of the cross section of the flushing device 200, which depicts one of the openings 220 in the wall 218. FIG. 2E illustrates the free movement of the flaps 208, which are exposing the openings 220.

FIGS. 2F-2H illustrate the flushing device 200 in various stages of use. In FIG. 2F, the fluid 210 is flowing (from the fluid source) into the inlet port 202 and fills the flow chamber 206. The fluid 210 flows through the spike 15 of the drip chamber 14 and into the drip chamber 14 to the rest of the IV line. In particular, the fluid 210 in the flow chamber 206 enters the opening 17 of the spike 15 and flows through the lumen 19 of the spike 15 to enter the drip chamber 14. At this stage, because the flow chamber 206 is full of medical fluid 210 (and the fluid source presumably contains more medical fluid 210 than the saline chamber 212 contains saline 214), the pressure in the flow chamber 206 is greater than the pressure in the saline chamber 212. The pressure from the fluid 210 pushes the flaps 208 against the wall 218 and closes the openings 220. Consequently, saline 214 does not enter the flow chamber 206.

In FIG. 2G, the flow chamber 206 does not contain any fluid 210, which would happen when the fluid source is empty (and the fluid delivery is complete). At this stage, the pressure in the flow chamber 206 is less than the pressure in the saline chamber 212, which causes the saline 212 to move towards the openings 220 in the wall 218. The saline 212 pushes open the flaps 208 and enters the flow chamber 206. The saline 212 then flows through the spike 15 and into the drip chamber 14, which is shown in FIG. 2H.

FIGS. 2G and 2H illustrate the elastic qualities of the outer wall 252 of the flushing device 200 and the saline chamber 212. In FIG. 2G, when saline 212 is just starting to enter the flow chamber 206, the outer wall 252 begins to bow inwards (towards the flow chamber 206). As more saline 212 flows into the flow chamber 206 and out of the flushing device 200, the outer wall 252 bows inward more severely, as shown in FIG. 2H.

FIGS. 3A-3E illustrates a second example of the flushing device, in accordance with aspects of the present disclosure. As shown in FIG. 3A, the flushing device 300 can be coupled to an IV set between a medical fluid bag 12 and a drip chamber 14. The flushing device 300 has an inlet port 302 that fluidly connects the flushing device 300 to the medical fluid bag 12. In some embodiments, the inlet port 302 is a spike (depicted in FIG. 3B) configured to spike the medical fluid bag 12. The fluid 310 from the medical fluid bag 12 can flow through the inlet port 302, into the flushing device 300, and out the outlet port 304 on the other side of the flushing device 300. In the embodiment shown, the outlet port 304 is configured to receive a spike from a drip chamber 14. In other embodiments, the outlet port 304 can include a spike (e.g., for coupling the flushing device 300 directly to an IV tube 16). In embodiments where the outlet port 304 is a spike, the flushing device 300 can also include a cap covering the spike, where the cap is configured to be removed when the flushing device 300 is connected to the IV line.

FIG. 3B depicts the flushing device 300 before it is coupled to an IV set. In FIG. 3B, two of the walls of the flushing device 300 are transparent such that the features inside of the flushing device 300 are visible. The flushing device 300 has a flow chamber 306 and a saline chamber 312. As shown, the flow chamber 306 is adjacent to the saline chamber 312. In the embodiments shown in this disclosure, the flow chamber 306 and the saline chamber 312 are rectangular. However, other shapes are also possible. In some embodiments, the flow chamber 306 and the saline chamber 312 have different shapes.

The saline chamber 312 is separated from the flow chamber 306 by a wall 318. In accordance with the description of the wall, the opening(s), and the flap(s) described above, the wall 318 includes an opening 320 (shown in FIGS. 3D and 3E) that is covered by a flap 308. In the embodiment shown herein, the flap 308 constitutes the lower half of the wall 318, and the opening 320 extends across the entire width of the flushing device 300. In some embodiments, the flap 308 is a cutout portion of the wall 318, and the opening 320 occupies only part of the lower portion of the wall 318. In other embodiments, the flap 308 constitutes the lower half of the wall 318, and the opening 320 occupies only part of the lower portion of the wall 318. Optionally, the flap 308 constitutes the entire wall 318.

The saline chamber 312 comprises a saline inlet 354, which is configured to fluidly connect the saline chamber 312 to a saline source—e.g., saline source 356 shown in FIGS. 3A and 3C-3E. FIGS. 3A and 3C-3E also depict the clamp 358 coupled to the saline inlet 354. The clamp 358 has a slot with a notch on one end of the slot, where the slot and the notch are sized to fit around the saline inlet 354. A caregiver can slide the clamp 358 relative to the saline inlet 354 to control the flow of saline 314 from the saline source 356 to the saline chamber 312. The slot is narrower than the notch, so when the saline inlet 354 is inside the slot, the saline inlet 354 does not fluidly connect the saline source 356 to the saline chamber 312. On the other hand, when the saline inlet 354 is inside the notch, the saline inlet 354 is open, so saline 314 can leave the saline source 356 and fill the saline chamber 312. In use, the caregiver can use the clamp 358 to open the saline inlet 354 after the IV line has been primed and the delivery of the fluid 310 from the medical fluid bag 12 has started (but before the medical fluid bag 12 is emptied).

FIG. 3C illustrates a cross-section of the flushing device 300 when fluid 310 is being transported from the medical fluid bag 12 to the IV line and the patient. Fluid 310 flows from the medical fluid bag 12 and into the flow chamber 306 through the inlet 302. The fluid 310 then flows out the outlet 304 and into the IV line (i.e., the drip chamber, the IV tubing, or another fluid connector).

The flap 308 of the flushing device can be configured as a one-way valve to permit a fluid to move from the saline inlet 354 toward the flow chamber 306, and resist movement of a fluid from the flow chamber 306 toward the saline inlet 354. In some embodiments, as shown in FIGS. 3B-3E, the floor of the saline chamber 312 is slightly higher than the floor of the flow chamber 306, and, when the flap 308 is in the closed position (shown in FIGS. 3B and 3C), the bottom of the flap 308 rests against the step formed by the two floors. Consequently, the flap 308 is a one-way flap that can only swing into the flow chamber 306. At the stage of fluid delivery shown in FIG. 3C, the fluid 310 presses against the flap 308 and keeps the opening 320 closed, which restricts the flow of saline into the flow chamber.

When the fluid source 12 is empty, as depicted in FIG. 3D, the fluid 310 does not apply pressure to the flap 308. As a result, the saline 314 in the saline chamber 312 pushes against the flap 308 and exposes the opening 320. The saline 314 can then flow from the saline chamber 312 to the flow chamber 306. Stated differently, when the pressure in the flow chamber 306 is less than the pressure in the saline chamber 312, the saline 314 can open the flap 308 to expose the opening 320. After the saline 314 enters the saline chamber 312, the saline 314 flows out the outlet 304 and into the IV line to flush the IV line.

In some embodiments, the saline source 356 comprises a collapsible bag (e.g., a plastic bag) that collapses as the saline 314 is emptied out of the saline source 356. The collapsible bag can help lock the saline 314 in the IV line and limit the likelihood of backflow in the IV line.

With traditional methods and devices for flushing IV lines after fluid delivery, some of the medical fluid is wasted because when the medical fluid is at the same level as the saline (i.e., the height of the medical fluid in the IV set is the same as the height of the saline), the saline will begin to flow into the main delivery line and dilute the medical fluid. This wastes medical fluid and extends the time required to infuse the medical fluid. On the other hand, with the devices described herein, the saline does not enter the IV line until after all of the medical fluid enters the IV line.

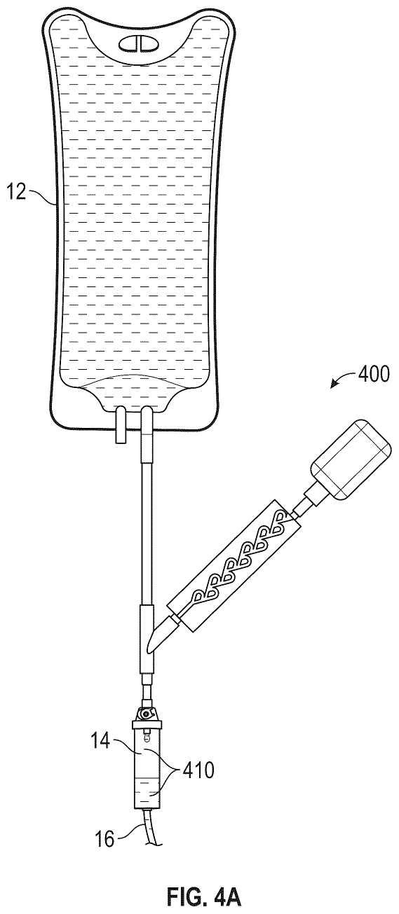

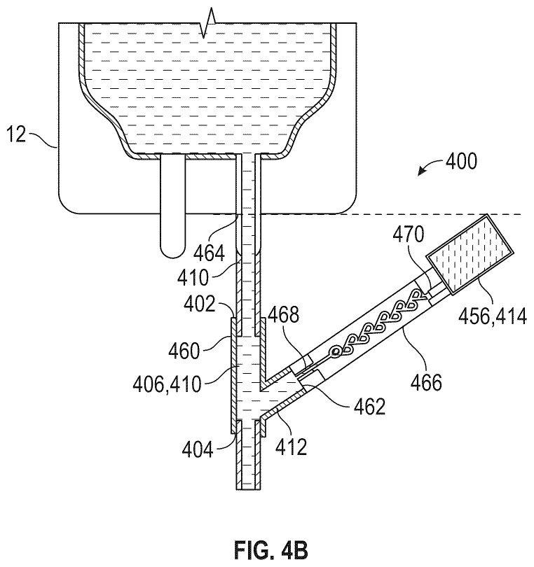

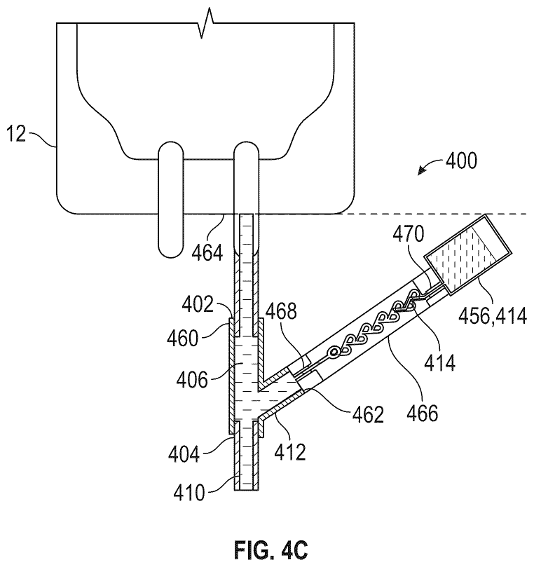

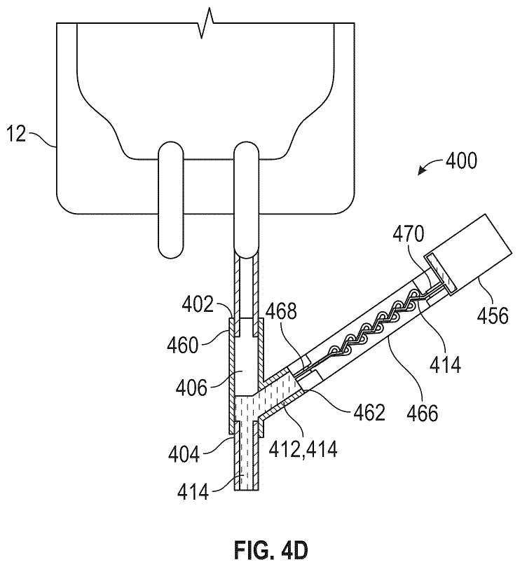

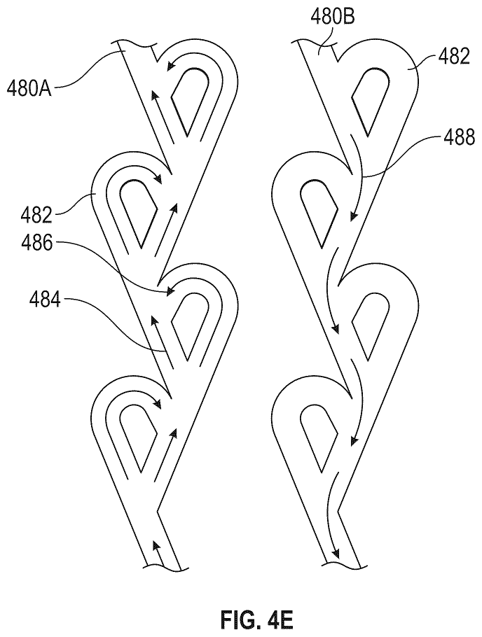

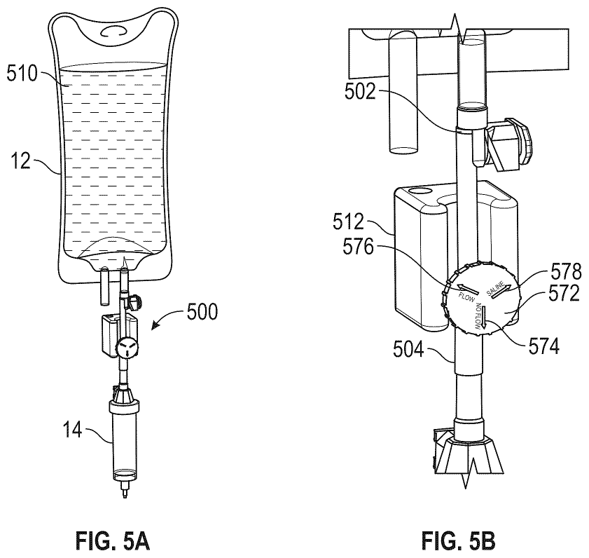

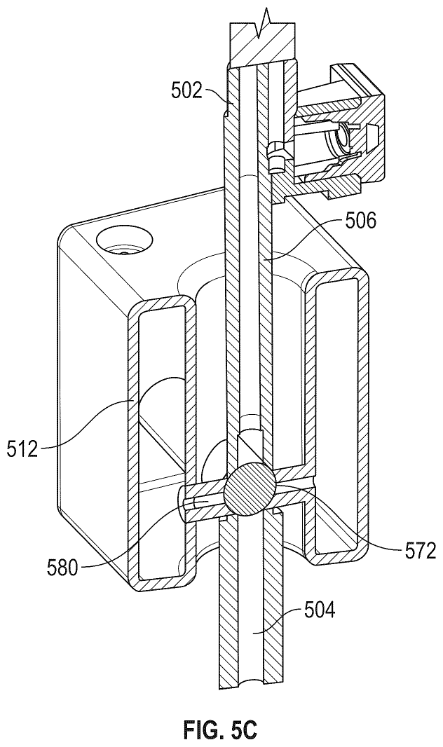

The flushing device 100 shown in FIG. 1 is not required to have a one-way flap that controls the flow of saline. Other configurations of the flushing device 100 are illustrated in FIGS. 4A-5B and described in greater detail below. FIGS. 4A-4E illustrate a third example of the flushing device, in accordance with aspects of the present disclosure. FIG. 4A demonstrates that the flushing device 400 can be coupled to an IV set between a medical fluid bag 12 and a drip chamber 14. The flushing device 400 comprises a Y-site connector 460, a Tesla valve 466 (discussed in greater detail below with respect to FIG. 4E) coupled to the Y-site connector 460, and, optionally, a saline source 456 coupled to the Tesla valve 466.

As shown in FIG. 4B, the Y-site connector 460 comprises two inlets that lead to two lumens, which converge and lead to an outlet. In particular, the Y-site connector 460 has a first inlet 402 that leads to a flow lumen 406 (which can also be referred to as a flow channel 406) and an outlet 404 at the opposite side (downstream) of the flow lumen 406. The Y-site connector 460 also has a second inlet 462 that leads to a saline lumen 412 (or a saline channel 412), which converges with the flow lumen 406 at point upstream of the outlet 404. The first inlet 402 is configured to couple to a fluid source, such as the medical fluid bag 12, and fluid 410 can flow from the medical fluid bag 12, into the first inlet 402, through the flow lumen 406, and out the outlet 404 to the IV line and the patient. The saline lumen 412 is configured to transport saline 414 from the saline source 456 and the Tesla valve 466, to the downstream end of the flow lumen 406, and into the IV line to flush the IV line after fluid delivery.

Before fluid delivery is complete, the saline 414 remains in the saline source 456. This is because the flushing device 400 is configured such that when the saline source 456 is coupled to the flushing device 400, the saline source 456 is at the same height 464 as the bottom of the medical fluid bag 12. (Optionally, the saline source 456 is below the bottom of the medical fluid bag 12.) Because of the pressure differences caused by relative heights and sizes of the saline source 456 and the medical fluid bag 12 (i.e., the medical fluid bag 12 is higher and, in most cases, larger than the saline source 456), the saline 414 will not flow out of the saline source 456 until the level of the fluid 410 is below the line 464. Consequently, the saline 414 will not flow out of the saline source 456 until the medical fluid bag 12 is empty.

In FIG. 4C, the medical fluid bag 12 is empty, and the level of the fluid 410 is at the line 464. At this stage, the saline 414 begins to flow out of the saline source 456, into the Tesla valve 466, and towards the saline lumen 412. Because of the pressure differential described above, the saline 414 automatically begins to flow at this point.

As will be discussed in greater detail below, the fluid path through the Tesla valve 466 is smaller than the flow lumen 406, such that the saline 414 will not reach the saline lumen 412 until after all of the fluid 410 flows out of the saline lumen 412. This prevents mixture of the saline 414 and the fluid 410 and, thus, dilution of the fluid 410.

In FIG. 4D, all of the fluid 410 has exited the flushing device 400, and only saline 414 remains. The saline 414 will flow out of the saline source 456 and the flushing device 400 until all of the saline 414 is in the IV line, which flushes the IV line with saline 414 after the delivery of the fluid 410.

Notably, because the medical fluid bag 12 is generally larger than the saline source 456, it is possible for the fluid 410 to flow up the saline lumen 412 and mix with the saline 414 in the saline source 456. For this reason, the flushing device 400 also includes a Tesla valve 466 in between the saline lumen 412 and the saline source 456. The Tesla valve 466 has a downstream end 468 that is coupled to the second inlet 462 and an upstream end 470 that is coupled to the saline source 456. The Tesla valve 466 restricts the flow of the fluid 410 into the saline source 456 and transports the saline 414 from the saline source 456 to the saline lumen 412 (after fluid delivery is complete).

FIG. 4E is a diagram that depicts the fluid path 480 inside of a Tesla valve (such as the Tesla valve 466 that is part of the flushing device 400) and fluid flow patterns 484, 486, 488 through a Tesla valve. As shown by FIG. 4E, the fluid path 480 is a repeating pattern of alternating recesses 482. The left fluid path 480A depicts an upstream flow of fluid (i.e., from the downstream end 468 adjacent to the saline lumen 412 towards the upstream end 470 that is coupled to the saline source 456 in FIGS. 4B-4D) through the Tesla valve. Before the first recess, the fluid diverges into two streams: the primary stream 484 and the secondary stream 486 through the recess 482. The fluid in the secondary stream 486 loses momentum as it goes around the bend of the recess 482. Subsequently, the fluid in the secondary stream 486 converges with the fluid in the primary stream 484, which obstructs the fluid as it flows upstream. Consequently, the Tesla valve restricts the flow of fluid upstream. In the context of the flushing device 400 in FIGS. 4B-4D, the Tesla valve 466 restricts the flow of fluid 410 towards the saline source 456, which limits the mixing of the fluid 410 and the saline 414. On the other hand, fluid can flow easily downstream (i.e., from the saline source 456 towards the saline lumen 412 in FIGS. 4B-4D) through the Tesla valve, as shown by the right fluid path 480B. Most of the fluid remains in the primary stream 488, which means there is little resistance in the Tesla valve in the downstream direction. Accordingly, with respect to the flushing device 400, the Tesla valve 466 facilitates the downstream flow of the saline 414 towards the saline lumen 412.

Another reason that the Tesla valve 466 restricts the flow of the fluid 410 into the saline source 456 is the relative diameters of the fluid path in the Tesla valve 466 and the flow lumen 406. Specifically, the flow lumen 406 (and the path between the Y-site connector 460 and the medical fluid bag 12) has a larger diameter than the fluid path in the Tesla valve 466. As a result, the fluid 410 travels up the Tesla valve 466 slower than the fluid 410 travels through the flow lumen 406 and towards the IV line.

FIGS. 5A-5C illustrate a fourth example of the flushing device, in accordance with aspects of the present disclosure. As shown in FIG. 5A, the flushing device 500 can be coupled to an IV set between a medical fluid bag 12 and a drip chamber 14. As shown in FIG. 5B, the flushing device 500 has an inlet port 502 that fluidly connects the flushing device 500 to the medical fluid bag 12. In some embodiments, the inlet port 502 is a spike configured to spike the medical fluid bag 12. The fluid 510 from the medical fluid bag 12 can flow through the inlet port 502, into the flushing device 500, and out the outlet port 504 on the other side of the flushing device 500. In the embodiment shown, the outlet port 504 is configured to receive a spike from a drip chamber 14. In other embodiments, the outlet port 504 can include a spike (e.g., for coupling the flushing device 500 directly to an IV tube).

Referring now to FIG. 5C, the flushing device 500 includes a flow channel 506 that can be fluidly connected to the inlet port 502 and the outlet port 504, depending on the setting of a dial 572, which is discussed in greater detail below. In some embodiments, the flow channel 506 is the channel or tube that extends between the inlet port 502 and the outlet port 504. The flushing device 500 also includes a saline chamber 512 filled with saline and fluidly connected to the flow channel 506 and the outlet port 504 via a saline channel 580. In some embodiments, the saline chamber 512 includes an opening. In FIGS. 5B and 5C, this opening is positioned at the top of the saline chamber 512. A caregiver can refill the saline chamber 512 with saline via the opening so that the flushing device 500 can be reused even after the saline chamber 512 is emptied.

The saline channel 580 is transverse to the flow channel 506 and intersects the flow channel 506 at an intersection point, and the dial 572 is positioned at this intersection point to control the fluid connection between the flow channel 506 and the saline channel 580. Consequently, the dial 572 also controls the fluid connection between the fluid source (i.e., the medical fluid bag 12 in FIG. 5A), the saline chamber 512, and the outlet port 504 (which ultimately leads to the patient). The dial 572 comprises three settings: a first (“no flow”) setting 574, a second (“flow”) setting 576, and a third (“saline”) setting 578. Thus, the dial 572 can be used by caregivers to switch the flushing device 500 between different workflow modes.

In some embodiments of the present disclosure, the dial 572 is a three-way valve. For example, the dial 572 can have a connecting channel that fluidly connects or obstructs the flow channel 506, the saline channel 580, and the outlet port 504 based on the setting to which the dial 572 is turned.

When the dial 572 is set to “no flow” 574, the dial 572 obstructs or resists fluid flow from the flow channel 506 toward the outlet port 504 and from the saline channel 580 toward the outlet port 504. This can be accomplished by misaligning the connecting channel relative to both the flow channel 506 and the saline channel 580. Thus, both the fluid 510 from the fluid source 12 and the saline from the saline chamber 512 are blocked from moving toward the outlet port 504. The “no flow” setting 574 can be useful before the flushing device 500 is coupled to the medical fluid bag 12 and the drip chamber 14. For instance, the caregiver might want to use the “no flow” setting 574 when spiking the medical fluid bag 12 with the flushing device 500 to prevent the fluid 410 and the saline from spilling out of the flushing device 500 before fluid delivery begins.

When the dial 572 is set to “flow” 576, such as when it is time for fluid delivery, the dial 572 permits fluid flow from the flow channel 506 to the outlet port 504, while resisting or obstructing fluid flow from the saline channel 580 to the outlet port 504. This can be accomplished by aligning the connecting channel relative to the flow channel 506 and misaligning the connecting channel relative to the saline channel 580. Thus, the fluid 510 can flow through the flow channel 506 to the outlet port 504 and towards the patient. When the flushing device 500 is in the “flow” setting 576, saline is kept in the saline chamber 512 to limit mixing of the fluid 510 and the saline.

When the dial 572 is set to “saline” 578, such as when all the fluid 510 is in the IV line (i.e., the medical fluid bag 12 is empty), the dial 572 permits fluid flow from the saline chamber 512 to the outlet 504, and the dial 572 resists or obstructs fluid flow from the flow channel 506 to the outlet port 504. This can be accomplished by misaligning the connecting channel relative to the flow channel 506 and aligning the connecting channel relative to the saline channel 580, thereby fluidly connecting the saline channel 580 and the outlet 504. Thus, when the dial 572 can be set to “saline” 578, the saline channel 580 is opened so that the saline can leave the saline chamber 512 and flow towards the flow channel 506 and the outlet 504. The caregiver can use the “saline” setting 578 after all of the medical fluid 510 has entered the IV line to flush the IV line with saline after fluid delivery. In a conventional flushing workflow, some of the medical fluid is diluted with saline and takes longer to be infused. However, with the flushing devices described herein, the saline does not enter the IV line until after the full dose of medical fluid is in the IV line, which limits dilution and medical fluid waste. Optionally, a caregiver can also use the “saline” setting 578 to prime the IV line before medical fluid delivery.

While the present disclosure describes use of the flushing device with a liquid drug and saline, the flushing devices described herein can also be used to facilitate consecutive delivery of two liquid drugs. For example, in any of the embodiments described herein, the saline source can be replaced with a fluid source containing a different medication. As a result, after the first fluid source containing the first medication is empty, the flushing device will facilitate the flow of the second medication from the second fluid source into the IV line.

### Illustration of Subject Technology As Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. A flushing device for an IV line, the flushing device comprising an inlet port configured to fluidly connect the flushing device to a fluid source, an outlet port configured to fluidly connect the flushing device to the IV line, a flow chamber fluidly connected to the inlet port and the outlet port, a saline chamber separated from the flow chamber by a wall and configured to be filled with saline, one or more openings in the wall, and one or more flaps overlapping each of the one or more openings from inside the flow chamber and movably coupled to the wall, wherein the one or more flaps close each of the one or more openings when a flow chamber pressure is greater than a saline chamber pressure, wherein the one or more flaps expose each of the one or more openings when the flow chamber pressure is less than the saline chamber pressure, such that each of the one or more openings fluidly connects the flow chamber and the saline chamber.

Clause 2. The flushing device of Clause 1, wherein the outlet port is configured to be coupled to a drip chamber of the IV line.

Clause 3. The flushing device of Clause 1, wherein each of the one or more openings is configured to facilitate a flow of the saline from the saline chamber to the flow chamber when the flow chamber pressure is less than the saline chamber pressure.

Clause 4. The flushing device of Clause 1, wherein the flow chamber pressure is less than the saline chamber pressure when the flow chamber is empty and the saline chamber contains at least some of the saline.

Clause 5. The flushing device of Clause 1, wherein the saline chamber is formed between the wall and an outer wall of the flushing device.

Clause 6. The flushing device of Clause 5, further comprising a stopper that presses the one or more flaps against the wall inside the flow chamber, wherein the stopper is slidably coupled to the wall such that the stopper is configured to slide towards the inlet port and away from the one or more flaps when the IV line is coupled to outlet port.

Clause 7. The flushing device of Clause 5, wherein the outer wall comprises an elastic material such that the outer wall is configured to bow inwards towards the flow chamber when the flow chamber pressure is less than the saline chamber pressure.

Clause 8. The flushing device of Clause 1, wherein the flow chamber is adjacent to the saline chamber.

Clause 9. The flushing device of Clause 8, further comprising a saline inlet in the saline chamber and a saline source filled with saline, wherein the saline inlet is configured to fluidly connect the saline source to the saline chamber.

Clause 10. The flushing device of Clause 9, wherein the saline source comprises a collapsible bag that is configured to collapse as the saline flows from the saline source to the saline chamber when the flow chamber pressure is less than the saline chamber pressure.

Clause 11. The flushing device of Clause 9, further comprising a clamp coupled to the saline inlet, wherein the clamp is configured to fluidly disconnect the saline source and the saline chamber when the IV line is being primed.

Clause 12. A flushing device for an IV line, the flushing device comprising: a Y-site connector comprising a first inlet that leads to a flow lumen and is configured to couple with a fluid source and a second inlet that leads to a saline lumen that intersects the flow lumen; and a Tesla valve comprising a downstream end coupled to the second inlet and an upstream end configured to couple with a saline source, wherein the flow lumen is configured to transport fluid from the fluid source to the IV line, wherein the Tesla valve is configured to obstruct a flow of the fluid towards the saline source and limit mixing of the fluid and the saline.

Clause 13. The flushing device of Clause 12, further comprising the saline source, wherein the saline source is configured to be positioned at a first height, the fluid source is configured to be positioned at a second height, and the first height is at or below the second height, such that the saline flows through the Tesla valve when a level of the fluid is at or below the first height.

Clause 14. The flushing device of Clause 12, wherein the saline lumen is configured to transport saline from the saline source to the IV line after the fluid source is empty.

Clause 15. The flushing device of Clause 12, wherein the flow lumen has a first diameter, a flow path in the Tesla valve has a second diameter, and the first diameter is larger than the second diameter, such that the Tesla valve is configured to slow the flow of the saline towards the IV line.

Clause 16. A flushing device for an IV line, the flushing device comprising an inlet port configured to fluidly connect the flushing device to a fluid source, an outlet port configured to fluidly connect the flushing device to the IV line, a flow channel fluidly connected to the inlet port and the outlet port, a saline channel fluidly connected to the flow channel at an intersection, a saline chamber fluidly connected to the saline channel, and a dial coupled to the flow channel and the saline channel at the intersection and comprising a no flow setting, a flow setting, and a saline setting, wherein when the dial is turned to the no flow setting, the dial obstructs the flow channel to resist fluid from the fluid source from flowing through the flow channel and obstructs the saline channel to resist saline from the saline chamber from flowing into the flow channel, wherein when the dial is turned to the flow setting, the dial permits the fluid from the fluid source to flow through the flow channel towards the outlet port and obstructs the saline channel to resist the saline from flowing into the flow channel, wherein when the dial is turned to the saline setting, the dial obstructs the flow channel to resist the fluid from the fluid source from flowing through the flow channel towards the outlet port and permits the saline to flow from the saline channel into the flow channel towards the outlet port.

Clause 17. The flushing device of Clause 16, wherein the dial comprises a connecting channel.

Clause 18. The flushing device of Clause 17, wherein, when the dial is turned to the no flow setting, the connecting channel is misaligned with the flow channel and the saline channel.

Clause 19. The flushing device of Clause 17, wherein, when the dial is turned to the flow setting, the connecting channel fluidly connects the flow channel and the outlet port.

Clause 20. The flushing device of Clause 17, wherein, when the dial is turned to the saline setting, the connecting channel fluidly connects the saline channel and the outlet port.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear,” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A flushing device for an intravenous (IV) line, the flushing device comprising an inlet port configured to fluidly connect the flushing device to a fluid source, an outlet port configured to fluidly connect the flushing device to the IV line, a flow chamber fluidly connected to the inlet port and the outlet port, a saline chamber separated from the flow chamber by a wall and configured to be filled with saline, one or more openings in the wall, and one or more flaps overlapping each of the one or more openings from inside the flow chamber and movably coupled to the wall,
wherein the one or more flaps close each of the one or more openings when a flow chamber pressure is greater than a saline chamber pressure,
wherein the one or more flaps expose each of the one or more openings when the flow chamber pressure is less than the saline chamber pressure, such that each of the one or more openings fluidly connects the flow chamber and the saline chamber.

2. The flushing device of claim 1, wherein the outlet port is configured to be coupled to a drip chamber of the IV line.

3. The flushing device of claim 1, wherein each of the one or more openings is configured to facilitate a flow of the saline from the saline chamber to the flow chamber when the flow chamber pressure is less than the saline chamber pressure.

4. The flushing device of claim 1, wherein the flow chamber pressure is less than the saline chamber pressure when the flow chamber is empty and the saline chamber contains at least some of the saline.

5. The flushing device of claim 1, wherein the saline chamber is formed between the wall and an outer wall of the flushing device.

6. The flushing device of claim 5, further comprising a stopper that presses the one or more flaps against the wall inside the flow chamber, wherein the stopper is slidably coupled to the wall such that the stopper is configured to slide towards the inlet port and away from the one or more flaps when the IV line is coupled to outlet port.

7. The flushing device of claim 5, wherein the outer wall comprises an elastic material such that the outer wall is configured to bow inwards towards the flow chamber when the flow chamber pressure is less than the saline chamber pressure.

8. The flushing device of claim 1, wherein the flow chamber is adjacent to the saline chamber.

9. The flushing device of claim 8, further comprising a saline inlet in the saline chamber and a saline source filled with saline, wherein the saline inlet is configured to fluidly connect the saline source to the saline chamber.

10. The flushing device of claim 9, wherein the saline source comprises a collapsible bag that is configured to collapse as the saline flows from the saline source to the saline chamber when the flow chamber pressure is less than the saline chamber pressure.

11. The flushing device of claim 9, further comprising a clamp coupled to the saline inlet, wherein the clamp is configured to fluidly disconnect the saline source and the saline chamber when the IV line is being primed.

12. A flushing device for an intravenous (IV) line, the flushing device comprising:
a Y-site connector comprising a first inlet that leads to a flow lumen and is configured to couple with a fluid source and a second inlet that leads to a saline lumen that intersects the flow lumen; and
a Tesla valve comprising a downstream end coupled to the second inlet and an upstream end configured to couple with a saline source,
wherein the flow lumen is configured to transport fluid from the fluid source to the IV line,
wherein the Tesla valve is configured to obstruct a flow of the fluid towards the saline source and limit mixing of the fluid and the saline.

13. The flushing device of claim 12, further comprising the saline source, wherein the saline source is configured to be positioned at a first height, the fluid source is configured to be positioned at a second height, and the first height is at or below the second height, such that the saline flows through the Tesla valve when a level of the fluid is at or below the first height.

14. The flushing device of claim 12, wherein the saline lumen is configured to transport saline from the saline source to the IV line after the fluid source is empty.

15. The flushing device of claim 12, wherein the flow lumen has a first diameter, a flow path in the Tesla valve has a second diameter, and the first diameter is larger than the second diameter, such that the Tesla valve is configured to slow the flow of the saline towards the IV line.

16. A flushing device for an intravenous (IV) line, the flushing device comprising an inlet port configured to fluidly connect the flushing device to a fluid source, an outlet port configured to fluidly connect the flushing device to the IV line, a flow channel fluidly connected to the inlet port and the outlet port, a saline channel fluidly connected to the flow channel at an intersection, a saline chamber fluidly connected to the saline channel, and a dial coupled to the flow channel and the saline channel at the intersection and comprising a no flow setting, a flow setting, and a saline setting,
wherein when the dial is turned to the no flow setting, the dial obstructs the flow channel to resist fluid from the fluid source from flowing through the flow channel and obstructs the saline channel to resist saline from the saline chamber from flowing into the flow channel,
wherein when the dial is turned to the flow setting, the dial permits the fluid from the fluid source to flow through the flow channel towards the outlet port and obstructs the saline channel to resist the saline from flowing into the flow channel,
wherein when the dial is turned to the saline setting, the dial obstructs the flow channel to resist the fluid from the fluid source from flowing through the flow channel towards the outlet port and permits the saline to flow from the saline channel into the flow channel towards the outlet port.

17. The flushing device of claim 16, wherein the dial comprises a connecting channel.

18. The flushing device of claim 17, wherein, when the dial is turned to the no flow setting, the connecting channel is misaligned with the flow channel and the saline channel.

19. The flushing device of claim 17, wherein, when the dial is turned to the flow setting, the connecting channel fluidly connects the flow channel and the outlet port.

20. The flushing device of claim 17, wherein, when the dial is turned to the saline setting, the connecting channel fluidly connects the saline channel and the outlet port.
